(19) United States
(12) Patent Application Publication
(10) Pub. No.: US 2024/0179285 A1
(43) Pub. Date: May 30, 2024

(54) PROCESSING DEVICE AND PROCESSING METHOD

(71) Applicant: CANON KABUSHIKI KAISHA, Tokyo (JP)

(72) Inventor: MASAKI ISHII, Kanagawa (JP)

(21) Appl. No.: 18/518,320

(22) Filed: Nov. 22, 2023

(30) Foreign Application Priority Data
Nov. 28, 2022 (JP) 2022-189018

(51) Int. Cl. H04N 13/144; A61B 5/00; A61B 5/16; H04N 13/128; H04N 13/366; H04N 23/68

(52) U.S. Cl. CPC H04N 13/144; A61B 5/163; A61B 5/7275; H04N 13/128; H04N 13/366; H04N 23/683; A61B 2503/12; H04N 2213/002

(57) ABSTRACT

A processing device acquires a moving image that includes frames in each of which a first image for display to a right eye of a user, and a second image for display to a left eye of the user. The processing device determines a likelihood of the user experiencing a particular symptom due to viewing the moving image, on a basis of information of contents of the moving image, information of the user, and characteristics of the moving image.

### BACKGROUND

### Field of the Disclosure

The present disclosure relates to a processing device and a processing method for performing processing relating to moving images.

### Description of the Related Art

In recent years, there is interest in image-capturing devices for acquiring photographs or moving images that can be stereoscopically viewed. There is also interest in display devices for viewing virtual reality (VR) moving images that have a high level of immersivity and a high level of realism. However, in a case of wearing a head-mounted display (HMD) or the like and viewing VR moving images, users may experience fatigue, VR sickness, or the like.

Methods for determining the likelihood of experiencing symptoms such as fatigue, VR sickness, or the like, alleviating these symptoms, and so forth, have been proposed. Japanese Patent Application Publication No. 2021-69045 discloses a device and a method for lowering a degree of contrast ratio of VR moving images in accordance with user age, in order to alleviate eyestrain and discomfort of the user. Also, Japanese Patent Application Publication No. 2011-193461 discloses a device for adjusting the amount of disparity in stereoscopic images in accordance with the time of day at which the stereoscopic images are broadcast, and the genre, which includes drama, documentary, baseball, soccer, or the like, thereof, in order to alleviate fatigue of the eyes of the user viewing stereoscopic images.

For example, a case will be assumed in which there are two VR moving images that are moving images of which the genre is “documentary”, one moving image being a moving image of scenery of a city shot from a vehicle, which is automobile, train, or the like, and the other moving image being a moving image of a vegetable field shot from a fixed point. In this case, it may be likely that users viewing the moving image of the scenery of the city shot from the vehicle will experience VR sickness. Conversely, it may be likely that the same users viewing the moving image shot from the fixed point will not experience VR sickness. However, the technology disclosed in Japanese Patent Application Publication Nos. 2021-69045 and 2011-193461 only uses the age of the user, the genre of the VR moving image, and so forth, and accordingly the likelihood of experiencing symptoms such as VR sickness, fatigue, or the like, from such two moving images, cannot be appropriately determined.

### SUMMARY

Accordingly, aspects of the present disclosure provide technology that enables the likelihood of particular symptoms being experienced when a user views a VR moving image to be determined.

An aspect of the disclosure is a processing device including a processor, and a memory storing a program which, when executed by the processor, causes the processing device to acquire a moving image that includes frames in each of which a first image for display to a right eye of a user, and a second image for display to a left eye of the user, and determine a likelihood of the user experiencing a particular symptom due to viewing the moving image, on a basis of information of contents of the moving image, information of the user, and characteristics of the moving image.

An aspect of the disclosure is a processing method, including acquiring a moving image that includes frames in each of which a first image for display to a right eye of a user, and a second image for display to a left eye of the user, and determining a likelihood of the user experiencing a particular symptom due to viewing the moving image, on a basis of information of contents of the moving image, information of the user, and characteristics of the moving image.

Further features of the present disclosure will become apparent from the following description of embodiments with reference to the attached drawings.

### BRIEF DESCRIPTION OF THE DRAWINGS

FIG. 1 is a block diagram of an image processing device according to a first embodiment.

FIG. 2 is a flowchart of image processing according to the first embodiment.

FIG. 3 is a diagram for describing determination criteria according to the first embodiment.

FIG. 4 is a diagram illustrating an example of detection limit and tolerance limit according to the first embodiment.

FIG. 5 is a diagram illustrating a graphical user interface (GUI) according to the first embodiment.

FIG. 6 is a flowchart of image processing according to a first modification.

### DESCRIPTION OF THE EMBODIMENTS

An embodiment of the present disclosure will be described below in detail with reference to the attached drawings.

In the following, a virtual reality (VR) image is an image that has a broader visual field range, angle of view, than a display range that can be displayed on a screen of a display unit at one time, such as VR 360° images, omnidirectional images, full-sphere images, or VR 180° images, hemisphere images. Such images have, for example, image ranges for a visual field up to 360 degrees in the vertical direction, which is vertical angle, angle from zenith, angle of elevation, angle of depression, altitude angle, pitch angle, and for a visual field up to up to 360° in the lateral direction, which is horizontal angle, azimuthal angle, yaw angle. VR 180° images have image ranges for a visual field up to 180° for each of these. Also, VR images include images that can be stereoscopically viewed by displaying two images with disparity side by side, and the user viewing the two images with the right and left eyes at the same time, enabling a sense of depth to be perceived. VR images in the present embodiment include moving images made up of a plurality of frames that are consecutive in time sequence, and live-view images, and are referred to as “VR moving images” in particular. Part of the range of VR images are displayed and reproduced on the screen thereof in accordance with a direction instructed by the user, causing change of the displayed image in real-time, along with movement of the body of the user, and accordingly, the user can experience a high level of realism and immersion.

In the present embodiment, technology will be described that enables the likelihood of the user experiencing symptoms such as fatigue, VR sickness, and so forth, when viewing a VR moving image (VR image), to be comprehended. Note that hereinafter, the user viewing VR moving images will be referred to as “viewing user”. Also, symptoms such as fatigue, VR sickness, and so forth, which are experienced due to viewing VR moving images, will be referred to as “VR symptoms”.

For example, in a case of the viewing user viewing moving-type contents, which is VR moving images shot from a camera placed in a vehicle, VR sickness, which is symptoms similar to motion sickness from being exposed to a VR environment, due to inconsistency between movement/change in the VR moving image and the movement of the body of the viewing user is readily experienced. Also, the viewing user may experience fatigue due to inconsistency in form, which is brightness, color, shape, or the like, of an object in a right-eye image, which is image displayed so as to be seen by the right eye, and in a left-eye image, which is image displayed so as to be seen by the left eye, in the VR moving image. In particular, in a case where inconsistency between the VR moving image and the movement of the viewing user, and inconsistency between the right-eye image and the left-eye image occur at the same time, the viewing user will be affected more greatly.

Now, special consideration needs to be given with respect to VR symptoms, regarding children still in growth, and those who are stereoscopically challenged. To this end, confirmation needs to be made prior to the viewing user viewing the VR moving image, regarding whether or not there is a likelihood of experiencing VR symptoms. However, someone needs to actually view the VR image in order to appropriately confirm/comprehend this likelihood, and there is a likelihood that the person performing this confirmation task will experience VR symptoms.

### First Embodiment

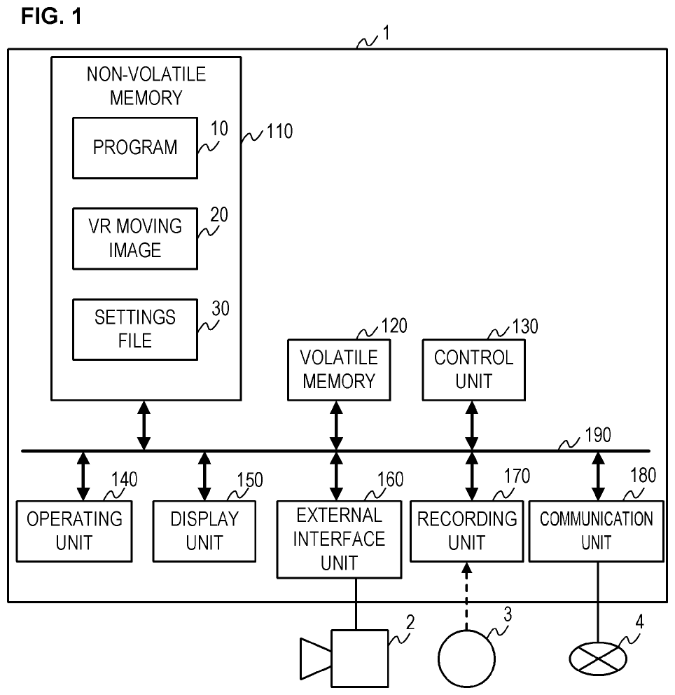

FIG. 1 is a block diagram illustrating a configuration of an image processing device 1 according to a first embodiment. The image processing device 1 is a personal computer (PC), a tablet terminal, a smartphone, or the like.

The image processing device 1 includes non-volatile memory 110, volatile memory 120, a control unit 130, an operating unit 140, a display unit 150, an external interface unit 160, a recording unit 170, and a communication unit 180. The components of the image processing device 1 are also connected to each other via a bus 190. Control signals and various types of data output from the control unit 130 are transmitted and received via the bus 190.

The non-volatile memory 110 has, for example, a hard disk drive (HDD) or the like. The non-volatile memory 110 stores programs 10, which are programs for causing the control unit 130 to execute various types of computation processing, VR moving images 20, a settings file 30, which is information indicating category of viewing user, computation processing results from the control unit 130, and so forth. The VR moving images 20 are VR moving image obtained by shooting a subject. Each VR moving image 20 includes a right-eye image, which is image displayed in front of the right eye, and a left-eye image, which is image displayed in front of the left eye.

The volatile memory 120 includes, for example, random-access memory (RAM) or the like. The volatile memory 120 is used to temporarily hold data. The programs 10 recorded in the non-volatile memory 110 or a recording medium 3 are temporarily loaded to the volatile memory 120 and executed by the control unit 130. Further, the volatile memory 120 is also used as working memory at the time of the control unit 130 executing computation processing.

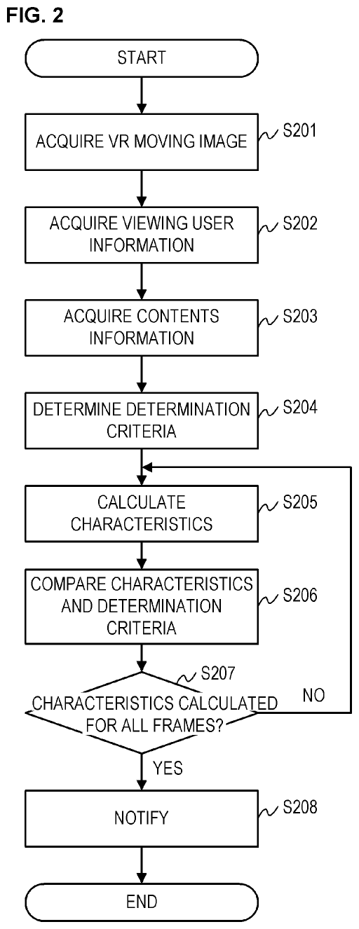

The control unit 130 controls the entire image processing device 1 following the program 10 loaded to the volatile memory 120. The control unit 130 is capable of central control of the image processing device 1. The control unit 130 can also execute processing of each step in an image processing method (see FIG. 2) that will be described later, by following the program. The control unit 130 includes a central processing unit (CPU) and so forth.

The operating unit 140 includes an operation member that a user can operate, for example keyboard, mouse, touchpad, or the like. The control unit 130 can control the components of the image processing device 1 in accordance with the contents of operations performed by the user at the operating unit 140.

The display unit 150 displays the VR moving images 20, and also displays a graphical user interface (GUI) screen or the like, including a GUI. The control unit 130 outputs controls signals to the components following the program. Accordingly, the control unit 130 controls the configurations of the image processing device 1 so as to generate/output video signals for display on the display unit 150. Note that the image processing device 1 may have an interface for outputting video signals for display on the display unit 150, instead of having the display unit 150. Accordingly, the display unit 150 may be an external monitor.

The external interface unit 160 communicates with an image-capturing device 2 connected to the image processing device 1. The image-capturing device 2 is, for example, a VR camera that acquires the VR moving images 20. The image processing device 1 and the image-capturing device 2 are connected by a wired connection using a Universal Serial Bus (USB) cable. Alternatively, the image processing device 1 and the image-capturing device 2 may be connected by a wireless connection using Bluetooth (registered trademark) or the like. The VR moving images 20 acquired by the image-capturing device 2 are stored in the non-volatile memory 110 via the external interface unit 160.

The recording unit 170 can read various types of data (programs 10 or VR moving images 20) and so forth, stored in the recording medium 3.

The communication unit 180 transmits various types of data (programs 10 or VR moving images 20) or the like to external equipment (equipment that the viewing user has) and so forth, via a network 4. For example, upon acquiring a VR moving image 20, external equipment such as a head-mounted display (HMD) or the like displays, out of the VR moving image 20 (image of frame corresponding to a playing point-in-time), a range corresponding to the attitude of the head of the viewing user. At this time, the external equipment displays the right-eye image of the VR moving image 20 so as to be visible from the right eye of the viewing user, and also displays the left-eye image of the VR moving image 20 so as to be visible from the left eye of the viewing user.

Note that description will be made in the first embodiment regarding a case where a recording medium (computer-readable recording medium) in which the programs 10 are stored is the non-volatile memory 110 or the recording medium 3. However, the programs 10 may be recorded in any recording medium as long as it is a computer-readable recording medium. For example, an external storage device or the like, omitted from illustration, may be used as the recording medium to provide the programs 10. Examples of the recording medium that can be used include flexible disks, hard disks, optical discs, magneto-optical discs, compact disc read-only memory (CD-ROM), compact disc-recordable (CD-R), magnetic tape, non-volatile memory (USB memory and so forth), read-only memory (ROM), and so forth. Also, the programs 10 may be supplied to the image processing device 1 via the network 4.

Image processing method according to the first embodiment will be described with reference to the flowchart in FIG. 2. Specifically, processing will be described regarding a case where a user that edits moving images (hereinafter, referred to as “editing user”) performs confirmation regarding whether or not there is a likelihood of a viewing user experiencing VR symptoms, which are symptoms such as fatigue, VR sickness, or the like, when viewing a VR moving image 20.

In step S201, the control unit 130 acquires a VR moving image 20 stored in the non-volatile memory 110. Alternatively, the control unit 130 may acquire a VR moving image 20 stored in another configuration (recording medium 3 or external device), as long as the VR moving image 20 can be acquired therefrom. For example, the control unit 130 may acquire a VR moving image 20 shot by an external device via the network 4.

In step S202, the control unit 130 acquires viewing user information indicating the category of the viewing user.

A case will be assumed, for example, in which one of the two categories of “under age 13 or stereoscopically challenged” and “other” can be set as the category of the viewing user. In this case, the control unit 130 acquires information indicating the category to which the viewing user falls under out of these two categories, as viewing user information. Specifically, when input is made by the editing user using the operating unit 140 regarding which of “under age 13 or stereoscopically challenged” and “other” the viewing user falls under, the control unit 130 acquires input results thereof as the viewing user information.

Note that instead of having the editing user input the category of the viewing user, the control unit 130 may acquire viewing user information from the settings file 30 in which is described the category of the viewing user. Also, instead of “under age 13”, a category may be set for a lower age such as “under age 6”, for example, or a category may be set indicating an aged person, such as “age 65 or above” or the like. Note that whether or not the viewing user is stereoscopically challenged can be determined from results of a Titmus stereo test of the viewing user, for example.

In step S203, the control unit 130 acquires contents information indicating the category of contents of the VR moving image 20. In the first embodiment, the contents information is information regarding what sort of situation that the VR moving image 20 was shot in, in other words, situation of shooting VR moving image 20. For example, a case will be assumed in which one of the three categories of “moving-type contents, which is VR moving images shot from a camera placed in a vehicle”, “camera shake contents, which is VR moving image shot in a state with camera shake occurring”, and “other” can be set as the category of the contents. In this case, the control unit 130 acquires the category to which the VR moving image 20 falls under out of these three categories, as contents information. Specifically, when input is made by the editing user using the operating unit 140 regarding which of “moving-type contents”, “camera shake contents”, and “other” the VR moving image 20 falls under, the control unit 130 acquires input results thereof as the contents information of the VR moving image 20. Note that moving-type contents and camera shake contents are contents in which VR sickness, visually induced motion sickness, is readily experienced.

Note that instead of having the editing user input the category of the contents, the control unit 130 may perform moving image analysis of the VR moving image 20, and determine whether or not the VR moving image 20 is “moving-type contents” or “camera shake contents”. Specifically, the control unit 130 calculates an optical flow of the VR moving image 20. When determining that the objects/the subjects in all images of a plurality of consecutive frames are moving in the same direction, on the basis of the optical flow, the control unit 130 can determine that the VR moving image 20 is “moving-type contents”. Conversely, when determining that movement of the objects in a first direction and movement of the objects in a second direction are being repeated in images of a plurality of frames that are consecutive, on the basis of the optical flow, the control unit 130 can determine that the VR moving image 20 is “camera shake contents”.

Note that it is sufficient for the viewing user information to be information relating to the viewing user. The viewing user information may be, for example, “information indicating age or generation such as date of birth” or “information relating to eyesight”. Also, it is sufficient for the contents information to be information relating to the contents of the VR moving image 20. The contents information may be, for example, information indicating which of a live-action moving image and an animation moving image that the VR moving image 20 falls under.

In step S204, the control unit 130 determines determination criteria for determining the likelihood of the viewing user experiencing VR symptoms, on the basis of the viewing user information and the contents information.

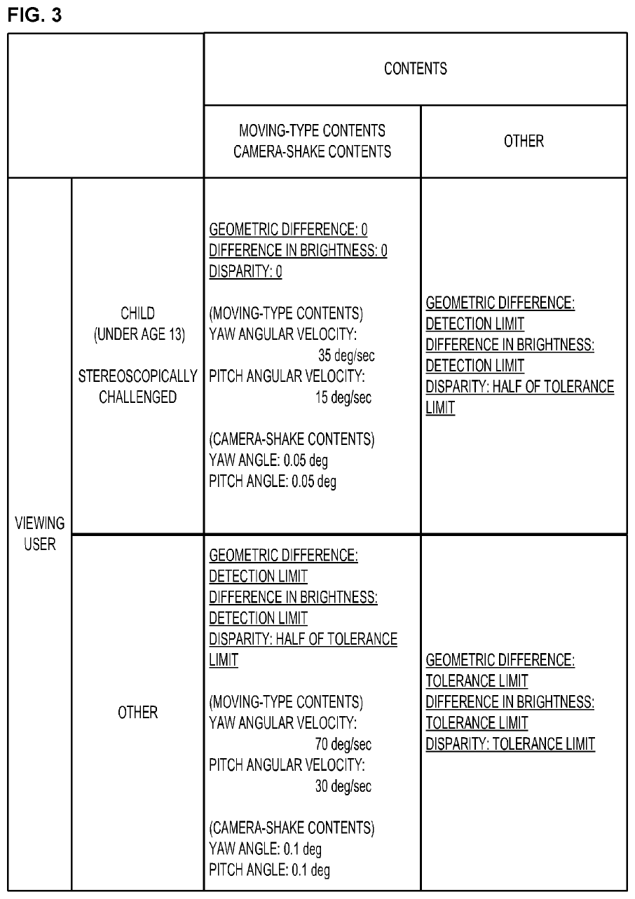

FIG. 3 is a diagram for describing the determination criteria according to the first embodiment. FIG. 3 shows that the determination criteria change in accordance with the category of the contents of the VR moving image 20, and the category of the viewing user. In a case where the viewing user is “child or stereoscopically challenged”, the determination criteria are set to stricter criteria in which determination is more readily made that the viewing user is likely to experience VR symptoms than in a case where the viewing user is “other”. Also, in a case where the contents are “moving-type contents or camera shake contents”, the determination criteria are set to stricter criteria than in a case where the contents are “other”.

Now, the determination criteria include criteria regarding binocular image characteristics, and criteria regarding contents characteristics. The binocular image characteristics are characteristics indicating difference (e.g. at least one of shift in size, shift in position, shift in rotation, difference in brightness, difference in color, difference in contrast ratio, disparity, and so forth) between the right-eye image and the left-eye image. The contents characteristics are characteristics indicating measure of movement (e.g. yaw angular velocity, pitch angular velocity, and so forth) of the image-capturing device that shot the VR moving image 20. Note that the contents characteristics may be characteristics indicating the measure of movement of a subject in the VR moving image 20.

For example, in a case of determining that the VR moving image 20 is “moving-type contents” and the viewing user is “under age 13”, the control unit 130 determines determination criteria for binocular image characteristics in which geometric difference, difference in brightness, and disparity, between the right-eye image and the left-eye image are all 0. Also, in this case, the control unit 130 determines determination criteria of contents characteristics in which the yaw angular velocity is 35 degrees/second, and also the pitch angular velocity is 15 degrees/second.

Also, in a case of determining that the VR moving image 20 is “other” and the viewing user is “under age 13”, for example, the control unit 130 determines determination criteria for binocular image characteristics in which geometric difference and difference in brightness are detection limit, and disparity is half of tolerance limit. Also, in this case, the control unit 130 determines determination criteria of contents characteristics in which any yaw angular velocity and pitch angular velocity, or yaw angle and pitch angle, is tolerable.

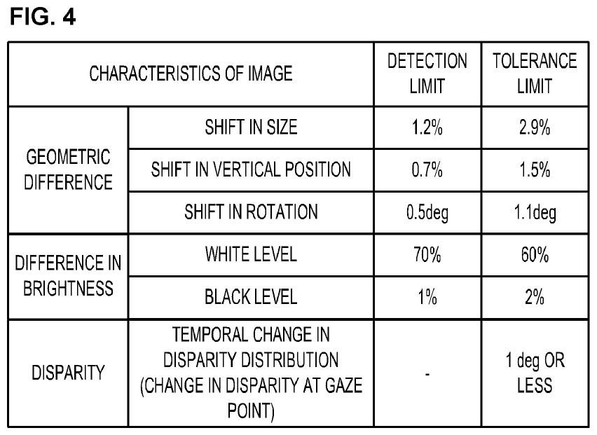

Now, detection limit is the limit of detection that humans can detect that a difference is present. Tolerance limit is the limit of difference that humans can tolerate. FIG. 4 is an example of detection limit and tolerance limit of difference between a right-eye image and a left-eye image. The example shown in FIG. 4 is disclosed in Non-Patent Literature (Takayuki Ito, “Research on 3D TV at NHK Science & Technology Research Laboratories (STRL)”, NHK STRL R&D 2010, No. 123, pp 48-55). The control unit 130 determines one out of the 2×2 criteria for a total of four criteria, shown in FIG. 3, as the determination criteria, on the basis of the contents information and the viewing user information acquired in steps S202 and S203.

Note that the determination criteria may further be determined on the basis of information of, for example, image quality (e.g. framerate, resolution, or the like) of the VR moving image 20. For example, the lower the framerate of the VR moving image 20 is, the greater the likelihood is that change in the display range of the VR moving image 20 will not be able to keep up with change in the attitude of the viewing user. Accordingly, the lower the framerate of the VR moving image 20 is, the more likely the viewing user is to experience VR sickness. Accordingly, in a case where the framerate of the VR moving image 20 is lower than a predetermined value, determination criteria the same as a case where the VR moving image 20 is “moving-type contents” or “camera shake contents” may be determined, even though the VR moving image 20 is “other than moving-type contents or the like”.

The processing of the following steps S205 and S206 is executed with respect to one frame of a VR moving image. Specifically, one frame of the VR moving image regarding which the processing of steps S205 and S206 is not executed yet is selected, and the processing of steps S205 and S206 is executed on this one frame. Hereinafter, the frame that is the object of the processing of steps S205 and S206 will be referred to as “object frame”.

In step S205, the control unit 130 calculates/acquires binocular image characteristics and contents characteristics of the image of the object frame. Specifically, the control unit 130 calculates difference (e.g. shift in size, shift in position, shift in rotation, difference in brightness, disparity, and so forth) between the right-eye image and the left-eye image, as binocular image characteristics. Also, when the VR moving image 20 is moving-type contents, the control unit 130 calculates the yaw angular velocity and the pitch angular velocity of the image-capturing device 2, as the contents characteristics. When the VR moving image 20 is camera shake contents, the control unit 130 calculates magnitude of camera shake, for example, yaw angle, pitch angle, and so forth, that is the amount of shake of the image-capturing device 2, as the contents characteristics.

Now, the control unit 130 can perform image analysis of the VR moving image 20, and calculate the yaw angular velocity and pitch angular velocity, or yaw angle and pitch angle, of the image-capturing device 2. Optical flow, which is technology for detecting movement of objects from a moving image, can be used for this image analysis. Specifically, the control unit 130 calculates the direction of movement, rotational direction, or speed, angular velocity, of the image-capturing device 2 by analyzing patterns in the way movement of objects appear between adjacent frames in the VR moving image 20, which occur due to the movement of the objects or the image-capturing device 2. Note that the control unit 130 may calculate the yaw angular velocity and pitch angular velocity, or yaw angle and pitch angle, of the image-capturing device 2 on the basis of values of an angular velocity sensor installed in the image-capturing device 2.

In step S206, the control unit 130 compares the binocular image characteristics of the object frame and the determination criteria for binocular image characteristics, and further compares the contents characteristics of the object frame and the determination criteria of contents characteristics. If one of the characteristics exceeds the determination criteria, the control unit 130 determines that there is a likelihood of the viewing user experiencing VR symptoms. In a case of determining that there is a likelihood of the viewing user experiencing VR symptoms, the control unit 130 registers the object frame in a notification list/warning list.

In step S207, the control unit 130 determines whether or not calculation of characteristics has been completed for all frames in the VR moving image 20. In a case of determining that calculation of characteristics has not been completed for all frames, the flow returns to step S205. In a case of determining that calculation of characteristics has been completed for all frames, the flow advances to step S208.

In step S208, the control unit 130 displays the calculated characteristics arrayed along with the right-eye image and the left-eye image. The control unit 130 also displays a graph representing time-series change of characteristics selected by the user. At this time, in a case where the VR moving image 20 contains a frame added to the notification list, the editing user is notified/warned that there is a likelihood that the viewing user will experience VT symptoms, which are symptoms such as fatigue, VR sickness, or the like.

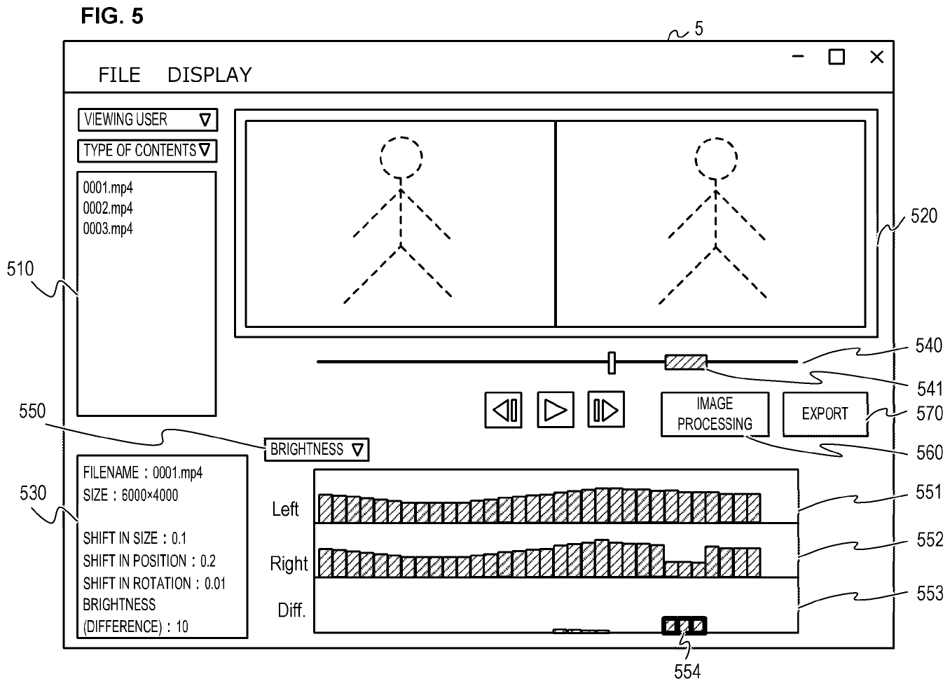

FIG. 5 is a schematic diagram illustrating an example of a GUI 5 that is displayed on the display unit 150 at the time of the editing user confirming the VR moving image 20. The GUI 5 includes a VR moving image list 510, a list for showing a plurality of VR moving images 20, a display area 520, an area for displaying the VR moving images 20, and a display area 530, area for displaying characteristics of the VR moving images 20. The GUI 5 also includes a bar 540, progress bar, showing a playing position, playing point-in-time, of the VR moving image 20, a pull-down menu 550 for the user to select characteristics, time-series graphs 551 to 553 of characteristics, and so forth.

The display area 520 displays images of a frame of the VR moving image 20 at a playing point-in-time that is specified at the bar 540. Note that right-eye image and left-eye image are displayed.

The display area 530 displays a filename and size of the VR moving image 20, and also displays the characteristics of the images of the frame corresponding to the playing point-in-time indicated by the bar 540. The time-series graphs 551 to 553 indicate the time-series change of the characteristics selected by the pull-down menu 550. The time-series graph 551 indicates the time-series change of characteristics of the left-eye image, and the time-series graph 552 indicates the time-series change of characteristics of the right-eye image. The time-series graph 553 indicates difference between the time-series graph 551 and the time-series graph 552 of the characteristics of the VR moving image 20, in other words, difference in characteristics between the right-eye image and the left-eye image.

Further, if a frame registered in the notification list (hereinafter referred to as “notified frame”) is present, the control unit 130 highlights and displays a section of the VR moving image 20 corresponding to the notified frame so as to be easily comprehended by the editing user. Specifically, in the GUI 5, a position/section 541 corresponding to the notified frame in the bar 540, and a position/section 554 corresponding to the notified frame in the time-series graph 553, are displayed enhanced. Further, in a case where the images of the frame displayed in the display area 520 are images of a notified frame, an outer frame of the display area 520 may be displayed enhanced. In this case, for example, the outer frame of the display area 520 may be set to a heavy frame, or the outer frame thereof may be displayed in red or the like.

The control unit 130 notifies the editing user that there is a likelihood that the viewing user may experience VR symptoms, thereby making an enhanced display of the position of the time-series graph 553 or the bar 540 corresponding to the notified frame. However, the control unit 130 may notify the editing user that there is a likelihood that the viewing user may experience VR symptoms by any method. For example, the control unit 130 may display a display item expressed by text on the display unit 150 to the effect that there is a likelihood that the viewing user may experience particular symptoms when viewing the VR moving image 20. The control unit 130 may notify the editing user that there is a likelihood that the viewing user may experience particular symptoms when viewing the VR moving image 20, by outputting audio.

As described above, according to the image processing device 1 of the first embodiment, the editing user can appropriately confirm the likelihood that the viewing user viewing a VR moving image will experience VR symptoms, which are symptoms such as fatigue, VR sickness, or the like, without actually viewing the VR moving image.

Note that the first embodiment is not limited to the above-described example, and the image processing device 1 may determine the likelihood of the viewing user experiencing VR symptoms, on the basis of information of contents of the VR moving image, information of the viewing user, and characteristics of the VR moving image, by any method. For example, a case will be assumed in which the image processing device 1 has a learning device, artificial intelligence, for machine learning. In this case, training of the learning device is performed in advance, by inputting information of a combination of the information of contents of the VR moving image, the information of the viewing user, and the characteristics of the VR moving image, and results of whether or not the viewing user experienced VR symptoms in a case of that combination, to the learning device for machine learning. In a case of performing determination, the image processing device 1 may input the combination of the information of contents of the VR moving image, the information of the viewing user, and the characteristics of the VR moving image, to the learning machine, thereby determining the likelihood of the viewing user experiencing VR symptoms.

### First Modification

Description will be made in a first modification regarding an image processing device 1 that performs, in addition to the processing according to the first embodiment, image processing (e.g. moving image editing) for reducing the likelihood of experiencing VR symptoms, which are symptoms such as fatigue, VR sickness, or the like.

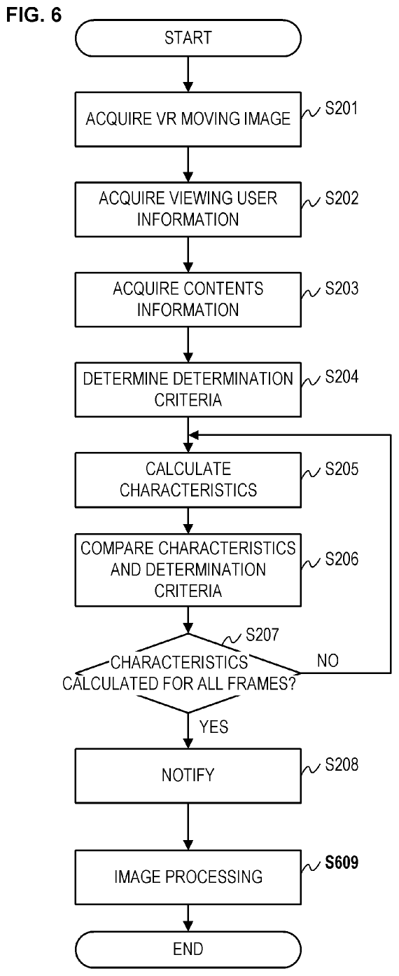

Image processing method according to the first modification will be described below with reference to FIG. 6. Note that the processing of steps S201 to S208 is the same as the processing of the steps denoted likewise in the first embodiment, and accordingly description thereof will be omitted.

In step S609, the control unit 130 executes image processing on the VR moving image 20 for reducing the likelihood of experiencing VR symptoms when viewing the VR moving image 20, in other words, for reducing VR symptoms. For example, for geometric shift between the right-eye image and the left-eye image, the control unit 130 calculates corresponding points between the right-eye image and the left-eye image, performs correction of scale and position of the right-eye image and the left-eye images so that the positions of the corresponding points in the two images agree with each other, and rotates these images. For difference in brightness between the right-eye image and the left-eye image, the control unit 130 calculates a luminance histogram for each of the right-eye image and the left-eye image, and corrects the right-eye image and the left-eye image so that the luminance histograms of the two agree, for example. The control unit 130 displays the VR moving image following correction in the display area 520. Finally, the control unit 130 saves the VR moving image 20 following correction in non-volatile memory.

Further, instead of correcting the VR moving image 20 in this way, the control unit 130 may remove/cut the frame registered in the notification list from the VR moving image 20 and generate a new VR moving image.

Note that the image processing in step S609 may be executed in a case where the editing user presses an image processing button 560 such as illustrated in FIG. 5. Saving of the file of the VR moving image 20 following correction may be executed in a case where the editing user presses an export button 570.

Also, the processing of step S609 may be executed only in a case where the VR moving image 20 contains a notified frame, or may be executed in any case.

As described above, according to the image processing device 1 of the first modification, the likelihood of experiencing VR symptoms, which are symptoms such as fatigue, VR sickness, or the like, when viewing VR moving images can be confirmed, and VR moving images in which the likelihood of experiencing VR symptoms is reduced can be generated.

According to the present disclosure, technology can be provided that enables the likelihood of particular symptoms being experienced when a user views a VR moving image to be determined more appropriately.

Also, in the above, “in a case where A is no less than B, the flow advances to step S1, and in a case where A is smaller/lower than B, the flow advances to step S2” may be reread as “in a case where A greater/higher than B, the flow advances to step S1, and in a case where A is not more than B, the flow advances to step S2”. Conversely, “in a case where A greater/higher than B, the flow advances to step S1, and in a case where A is not more than B, the flow advances to step S2” may be reread as “in a case where A is no less than B, the flow advances to step S1, and in a case where A is smaller/lower than B, the flow advances to step S2”. Accordingly, to the extent that no contradiction arises, the expression “no less than A” may be substituted with “A or greater, higher, longer or more, than A”, and may be reread as “greater, higher, longer or more, than A” and thus substituted. Conversely, the expression “not more than A” may be substituted with “A or smaller, lower, shorter or less than A”, and may be substituted with “smaller, lower, shorter or less than A” and thus reread. Also, “greater, higher, longer or more than A” may be reread as “no less than A”, and “smaller, lower, shorter of less than A” may be reread as “not more than A”.

Although the present disclosure has been described in detail above by way of preferred embodiments, the present disclosure is not limited to these particular embodiments, and various forms made without departing from the spirit and scope of the disclosure are encompassed by the present disclosure. Part of the above-described embodiments may be combined as appropriate.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the disclosure is not limited to the disclosed embodiments. The scope of the following claims is to be accorded the broadest interpretation so as to encompass all such modifications and equivalent structures and functions.

This application claims the benefit of priority from Japanese Patent Application No. 2022-189018, filed on Nov. 28, 2022, which is hereby incorporated by reference herein in its entirety.

What is claimed is:

1. A processing device comprising:
a processor; and
a memory storing a program which, when executed by the processor, causes the processing device to
acquire a moving image that includes frames in each of which a first image for display to a right eye of a user, and a second image for display to a left eye of the user, and
determine a likelihood of the user experiencing a particular symptom due to viewing the moving image, on a basis of information of contents of the moving image, information of the user, and characteristics of the moving image.

2. The processing device according to claim 1, wherein the program when executed by the processor causes the processing device to determine the likelihood of the user experiencing the particular symptom due to viewing the moving image, in accordance with a result of comparing criteria and the characteristics of the moving image, and
the criteria are in accordance with the information of contents of the moving image and the information of the user.

3. The processing device according to claim 1, wherein the information of the user is information based on at least one of age of the user and a state of stereoscopic abilities of the user.

4. The processing device according to claim 1, wherein the information of contents of the moving image is information relating to a situation at the time of shooting the moving image.

5. The processing device according to claim 4, wherein the information of contents of the moving image includes information of whether or not the moving image is shot by an image-capturing device placed in a vehicle.

6. The processing device according to claim 4, wherein the information of contents of the moving image includes information of whether or not the moving image is shot in a state of camera shake occurring.

7. The processing device according to claim 1, wherein the program when executed by the processor further causes the processing device to, in a case where it is determined that the user will likely experience the particular symptom, execute image processing on the moving image to reduce the likelihood that the user will experience the particular symptom.

8. The processing device according to claim 1, wherein the program when executed by the processor further causes the processing device to, in a case where it is determined that the user will likely experience the particular symptom, perform notification that the user will likely experience the particular symptom.

9. The processing device according to claim 8, wherein the program when executed by the processor causes the processing device to display the first image and the second image on a display, and also display the characteristics of the moving image.

10. The processing device according to claim 8, wherein the program when executed by the processor causes the processing device to display the first image and the second image on a display, and also display a graph indicating time sequence change of the characteristics of the moving image.

11. The processing device according to claim 8, wherein the program when executed by the processor causes the processing device to
determine a frame regarding which the user will likely experience the particular symptom, out of the moving image, and
in a case of determining that the user will likely experience the particular symptom, perform notification of a section of the frame regarding which the user will likely experience the particular symptom.

12. The processing device according to claim 11, wherein the program when executed by the processor causes the processing device to, in a case where it is determined that the user will likely experience the particular symptom, and where a graph indicating time sequence change of the characteristics of the moving image is displayed, enhance display of a position of the graph corresponding to the frame regarding which the user will likely experience the particular symptom.

13. The processing device according to claim 1, wherein the characteristics of the moving image include first characteristics indicating a difference between the first image and the second image, and second characteristics indicating a measure of movement of an image-capturing device that performed image-capturing of the moving image.

14. The processing device according to claim 13, wherein the first characteristics include at least one of shift in size, shift in position, shift in rotation, difference in brightness, difference in color, difference in contrast ratio, and disparity, between the first image and the second image.

15. The processing device according to claim 13, wherein, in a case where the moving image is contents shot by an image-capturing device placed in a vehicle, the second characteristics are a yaw angular velocity and a pitch angular velocity of the image-capturing device, and
in a case where the moving image is contents shot in a state of camera shake occurring, the second characteristics are magnitude of the camera shake.

16. The processing device according to claim 1, wherein the particular symptom is at least one of fatigue and sickness.

17. A processing method, comprising:
acquiring a moving image that includes frames in each of which a first image for display to a right eye of a user, and a second image for display to a left eye of the user; and
determining a likelihood of the user experiencing a particular symptom due to viewing the moving image, on a basis of information of contents of the moving image, information of the user, and characteristics of the moving image.

18. A non-transitory computer readable medium that stores a program, wherein
the program causes a computer to execute:
acquiring a moving image that includes frames in each of which a first image for display to a right eye of a user, and a second image for display to a left eye of the user; and
determining a likelihood of the user experiencing a particular symptom due to viewing the moving image, on a basis of information of contents of the moving image, information of the user, and characteristics of the moving image.
